(19) United States
(12) Patent Application Publication
(10) Pub. No.: US 2024/0010644 A1
(43) Pub. Date: Jan. 11, 2024

(54) PROCESS FOR THE PREPARATION OF ISAVUCONAZONIUM SULFATE

(71) Applicant: RK Pharma Inc., Pearl River, NY (US)

(72) Inventors: Ravishanker Kovi, North Brunswick, NJ (US); Jayaraman Kannappan, Vadodara (IN); Aditya N. Khanvilkar, Vadodara (IN); Jatin Kahar, Vadodara (IN); Suraj Chauhan, Vadodara (IN)

(21) Appl. No.: 18/218,610

(22) Filed: Jul. 6, 2023

(30) Foreign Application Priority Data
Jul. 6, 2022 (IN) 202221038870

(51) Int. Cl. C07D 417/14

(52) U.S. Cl. CPC C07D 417/14

(57) ABSTRACT

The present invention relates to an improved process for the preparation of Isavuconazonium sulfate. The present invention also provides a process for the preparation of key starting materials used for the preparation of Isavuconazonium sulfate.

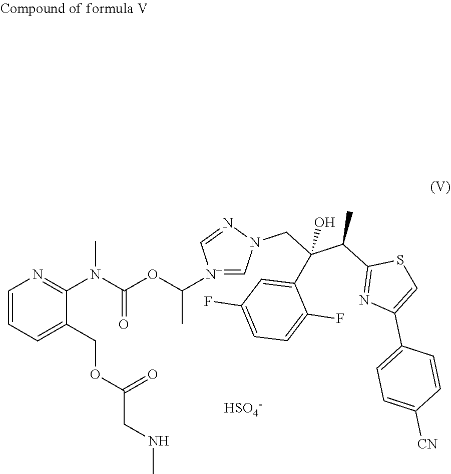

### RELATED APPLICATION

This application is a USA Complete application and claims priority to and the benefit of Indian Provisional Patent Application Number 202221038870 dated Jul. 6, 2022, the contents of which are incorporated herein in its entirety.

### FIELD OF THE INVENTION

The present invention relates to an improved process for the preparation of Isavuconazonium sulfate. The present invention also provides a process for the preparation of key starting materials used for the preparation of Isavuconazonium sulfate.

### BACKGROUND OF THE INVENTION

Isavuconazole is a broad-spectrum triazole and an alternative first-line agent in the treatment of pulmonary aspergillosis. Isavuconazole can be given orally or intravenously. After administration, the prodrug, Isavuconazonium, is cleaved by plasma esterase's to the active agent Isavuconazole.

Isavuconazole or Cresemba™ is the newest azole antifungal approved that comes as the salt formulation Isavuconazonium. Isavuconazonium sulfate marketed as CRESEMBA® is a capsule for oral administration. Isavuconazonium sulfate is also marketed as CRESEMBA® for injection for intravenous administration.

Isavuconazonium sulfate is chemically known 1-[[N-methyl-N-3-[(methylamino)acetoxymethyl] pyridin-2-yl] carbamoyloxy] ethyl-1-[(2R,3R)-2-(2,5-difluorophenyl)-2-hydroxy-3-[4-(4-cyanophenyl) thiazol-2-yl] butyl]-1H-[1,2,4]-triazo-4-ium Sulfate and is structurally represented by formula (V):

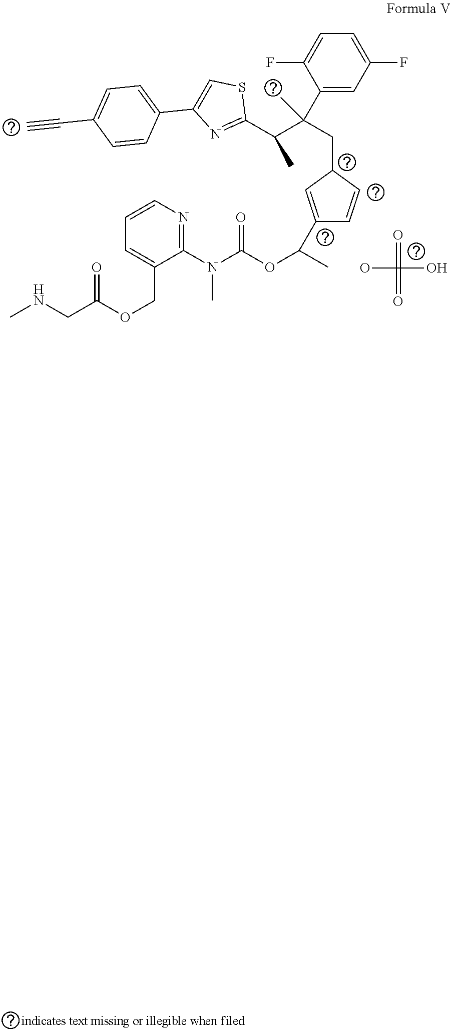

Isavuconazonium sulfate (BAL8557) is indicated for the treatment of antifungal infection. Isavuconazonium sulfate is a prodrug of Isavuconazole (BAL4815), which is chemically known 4-{2-[(1R,2R)-(2,5-Difluorophenyl)-2-hydroxy-1-methyl-3-(1H-1,2,4-triazol-1-yl) propyl]-1,3-thiazol-4-yl} benzo nitrile compound of Formula I.

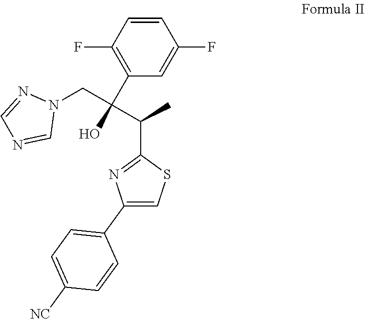 [SMILES: C[C@@H](c1nc(-c2ccc(C#N)cc2)cs1)[C@](O)(Cn1cncn1)c1cc(F)ccc1F]

U.S. Pat. No. 6,812,238 (referred to herein as '238); 7,189,858 (referred to herein as '858); 7,459,561 (referred to herein as '561) describe Isavuconazonium and its process for the preparation thereof.

The US Pat. '238 don't exemplify the process of preparation of Isavuconazonium sulfate. The said patent describes the process of preparation of Isavuconazonium chloride hydrochloride. Isavuconazonium sulfate is not prepared in anywhere in the reported literature.

The US Pat. '238 described the process for the Isavuconazonium or a pharmaceutically acceptable salt thereof, involves the condensation of Isavuconazonium and [N-Methyl-N-3((tert-butoxycarbonyl methylamino) acetoxymethyl) pyridine-2-yl] carbamic acid 1-chloro-ethyl ester.

The prior art reported process require almost 15-16 hours, whereas the present invention process requires only 2-4 hours. Inter alia prior art reported process requires too many step to prepare Isavuconazonium sulfate, whereas the present invention process requires fewer steps.

The inventors of the present invention surprisingly found that Isavuconazonium or a pharmaceutically acceptable salt thereof in high yield and high purity could be prepared by using substantially pure intermediates in suitable solvents.

Thus, an object of the present invention is to provide a process to overcome aforesaid problems and to provide simple, cost effective and industrially feasible processes for manufacture of Isavuconazonium sulfate.

Inventors of the present invention surprisingly also found that Isavuconazonium sulfate prepared from Isavuconazonium iodide hydrochloride, provides enhanced yield as well as purity as comparison to Isavuconazonium sulfate prepared from Isavuconazonium chloride hydrochloride.

The present invention provides an improved process for preparation of Isavuconazonium sulfate which provides better purity profile and which can be easily performed on industrial scale.

### Advantages of Present Invention Over Prior Art

1. Shorter reaction times.
2. Replacing expensive resins with low cost re-usable resins.
3. Higher yields and purities.
4. Reaction time is less and avoiding hazardous chemicals.
5. Easily operable, reproducible and non-tedious process.
6. Suitable for large scale synthesis.

### SUMMARY OF THE INVENTION

The present invention provides process for the preparation of Isavuconazonium sulfate, compound of formula V.

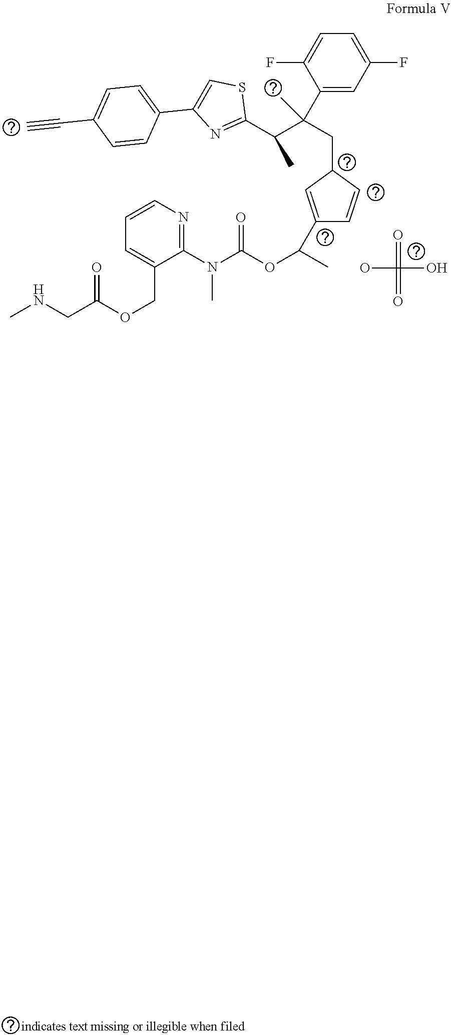

In one aspect, the present invention provides process for the preparation of compound of formula KSM-I.

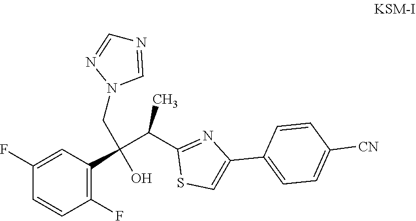 [SMILES: C[C@@H](c1nc(-c2ccc(C#N)cc2)cs1)[C@](O)(Cn1cncn1)c1cc(F)ccc1F]

Comprising, reacting a compound of formula 1, with a compound of formula 2 in presence of base under cyclization reaction to obtain a reaction mixture containing Isavuconazole, a compound of formula KSM-I.

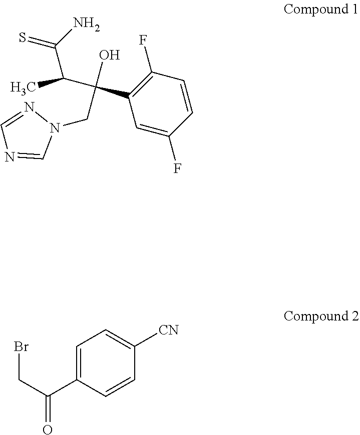 [SMILES: C[C@@H](C(N)=S)[C@](O)(Cn1cncn1)c1cc(F)ccc1F ; N#Cc1ccc(C(=O)CBr)cc1]

In second aspect, the present invention also provides a process for the preparation of compound of formula KSM-II.

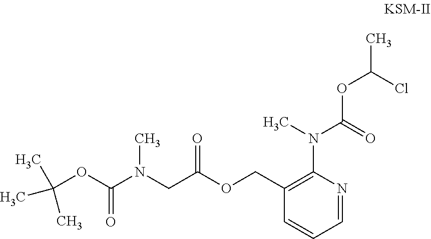 [SMILES: CC(Cl)OC(=O)N(C)c1ncccc1COC(=O)CN(C)C(=O)OC(C)(C)C]

In third aspect of the present invention, comprising reacting compound of formula 3, with a compound of formula 4 in presence of coupling reagent to obtain compound of formula 5. Further compound of formula 5 in presence of chloroethyl chloroformate to obtain compound of formula KSM-II.

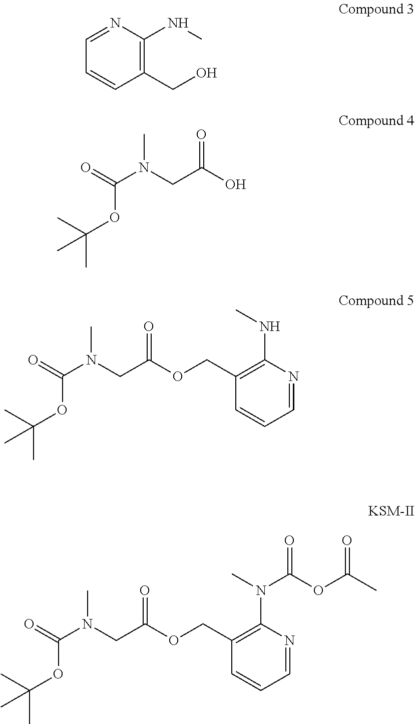

In a fourth aspect, reacting Isavuconazole, the compound of formula KSM-I obtained from the above reaction mixture with an ester of formula KSM-II in the presence of an iodide source and solvent to obtain N-Boc Isavuconazonium iodide;

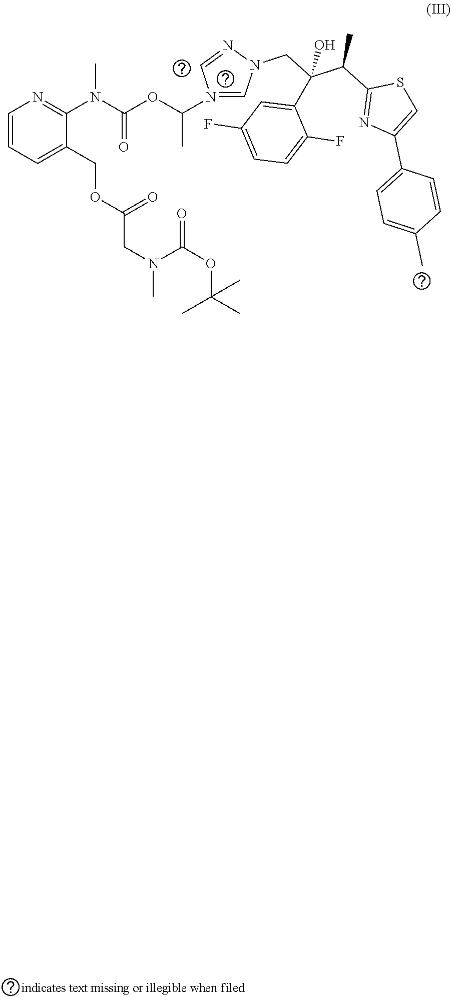

In a fifth aspect, converting N-Boc Isavuconazonium iodide to N-Boc Isavuconazonium hydrochloride; in sixth aspect, the present invention provides process for the preparation of N Boc Isavuconazonium hydro chloride by reacting N-Boc Isavuconazonium iodide treatment with a solvent ethyl acetate, and hydrochloride to obtain N-Boc Isavuconazonium hydro chloride with a purity of at least 95%; and converting N-Boc Isavuconazonium hydrochloride to Isavuconazonium sulfate.

In further aspect of the present invention conversion of Isavuconazonium iodide hydrochloride compound of formula IV in presence of Ion-exchange resin into Isavuconazonium sulfate salt. In another aspect, the present invention provides Isavuconazonium Sulfate, having purity greater than or equal to 99%.

### DETAILED DESCRIPTION OF THE INVENTION

The present invention provides a solid form of Isavuconazonium sulfate, a compound of formula V.

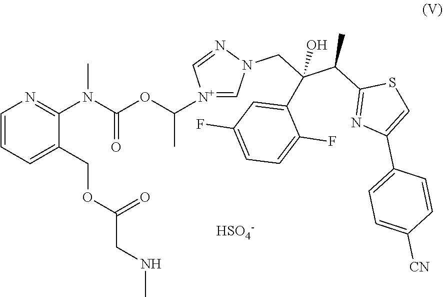

In an embodiment, the present invention provides Isavuconazonium sulfate, compound of Formula V, having purity more than 99% by as measured HPLC.

In one of the embodiment the Isavuconazonium Sulfate obtained by the process of present invention shown stability under standard condition of stability as described in United States Pharmacopoeia 2014.

In another embodiment of the present invention provides a process for the preparation of Isavuconazonium sulfate, a compound of formula V as illustrated in scheme 1.

### Schematic Representation of Scheme-1 is Depicted Below:

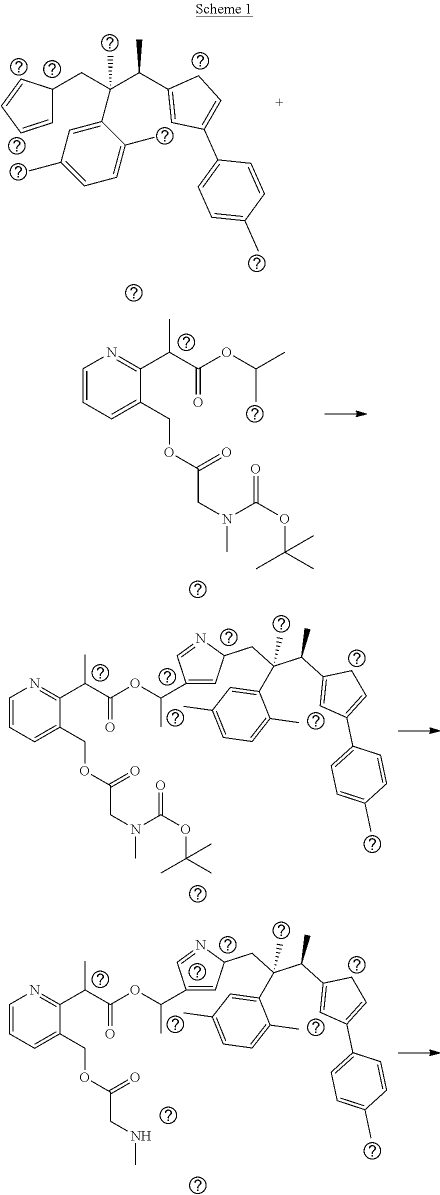

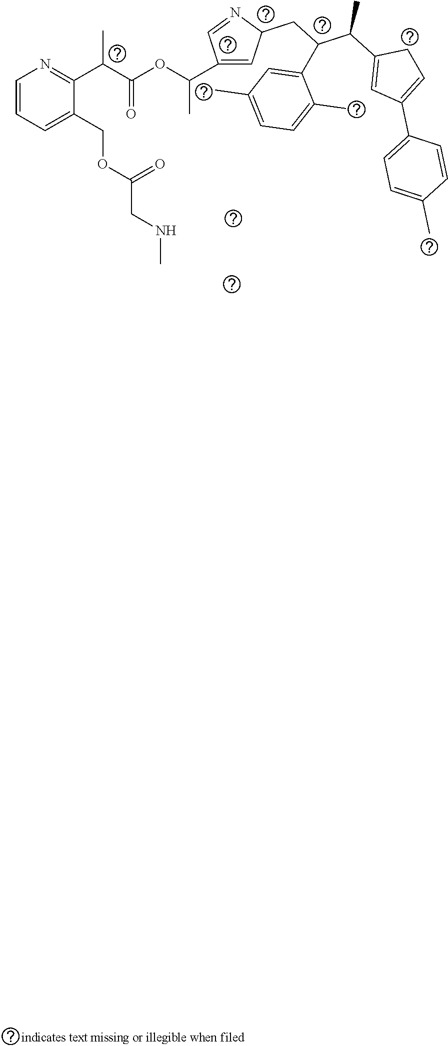 [SMILES: CNCC(=O)OCc1cccnc1C(C)C(=O)OC(C)C1=CC(CC(c2cc(C)ccc2C)[C@@H](C)C2=CC(c3ccc(C)cc3)=CC2)N=C1]

In one embodiment, the present invention provides a process for the preparation of Isavuconazonium sulfate, compound of Formula V.

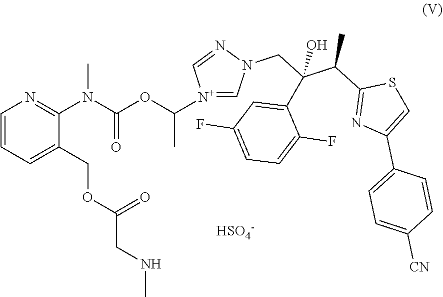

which includes steps of a) contacting Isavuconazonium iodide hydrochloride compound of Formula IV

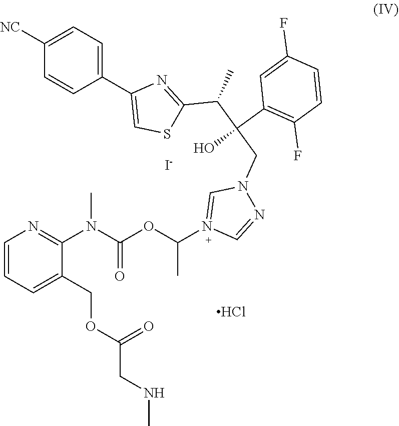

with anion exchange resin Diaion™ SA10A, isolation of Isavuconazonium sulfate in the suitable solvent.

Suitable solvent includes but are not limited to alcohol, halogenated solvent, acetates, ethers, water or mixture thereof. The alcohol such as methanol, ethanol, iso-propyl alcohol and the like; the halogenated solvent such dichloromethane; the ether such as methyl tert-butyl ether, ethyl tert-butyl ether, diethyl ether, di-tert-butyl ether and the acetate such as ethyl acetate, propyl acetate, butyl acetate and the like.

The anion exchange resin includes but are not limited to Diaion™ SA10A, Diaion™ SA11A, Diaion™ SA12A, Diaion™ NSA100, Diaion™ PA308, PA306, Amberjet 4000 Cl, Amberjet 4400 Cl and Amberjet 4600 Cl. Particularly Diaion™ SA10A.

In a second embodiment, the Isavuconazonium iodide hydrochloride compound of Formula IV, which includes steps of reacting Isavuconazole compound of formula KSM-I with [N-Methyl-N-3((tert-butoxycarbonylmethylamino) acetoxymethyl) pyridine-2-yl] carbamic acid 1-chloro-ethyl ester compound of formula KSM-II, in presence of iodide salt, in suitable solvent to obtain protected Isavuconazonium iodide compound of formula III. Iodide salt used for the reaction is potassium iodide or sodium iodide. Particularly Sodium iodide.

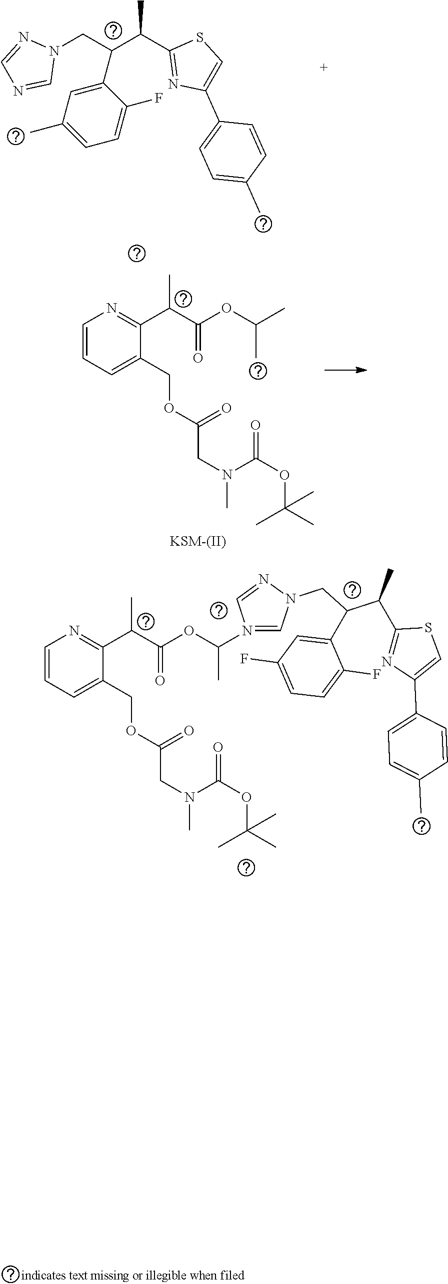

Suitable solvent used in the above step includes but are not limited to nitrile, ether, amides, alcohol, acetates, hydrocarbon, water or mixture thereof. The nitriles such as acetonitrile and propionitrile and the like; the ether such as methyl tert-butyl ether, ethyl tert-butyl ether, diethyl ether, di-tert-butyl ether, dioxane and the like; the amides such as dimethyl acetamide and dimethyl formamide and the like; the alcohol such as methanol, ethanol, isopropyl alcohol and the like; the acetate such as ethyl acetate, propyl acetate, butyl acetate and the like; hydrocarbon such as toluene, n-hexane, benzene, heptane and the like; In third embodiment, N-Boc Isavuconazonium iodide is converted to N-Boc Isavuconazonium hydro chloride using a pressure column containing chloride resin. the use of a pressure column in conversion of N-Boc Isavuconazonium iodide to N-Boc Isavuconazonium chloride, provides the N-Boc Isavuconazonium chloride to be obtained in constant yield.

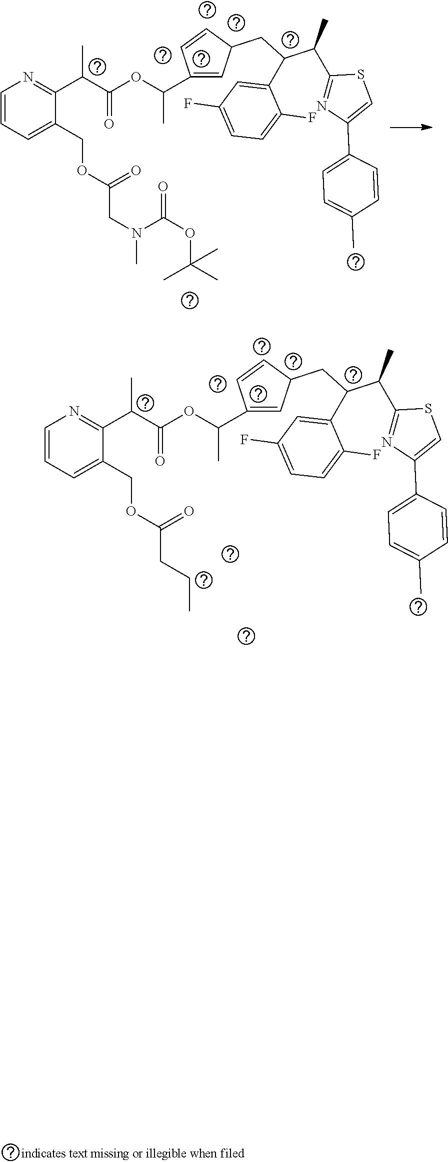 [SMILES: Cc1ccc(-c2csc([C@H](C)C(CC3C=CC(C(C)OC(=O)C(C)c4ncccc4COC(=O)CN(C)C(=O)OC(C)(C)C)=C3)c3cc(F)ccc3F)n2)cc1 ; CCCC(=O)OCc1cccnc1C(C)C(=O)OC(C)C1=CC(CC(c2cc(F)ccc2F)[C@@H](C)c2nc(-c3ccc(C)cc3)cs2)C=C1]

In a fourth embodiment, N-Boc Isavuconazonium chloride is isolated as a solid by a process comprising: i) removing the solvent from a solution comprising N-Boc Isavuconazonium chloride to obtain a residue; ii) contacting the residue with a mixture of ester and a hydrocarbon solvent to precipitate N-Boc Isavuconazonium chloride; and iii) isolating N-Boc Isavuconazonium chloride.

In fifth embodiment, the above process involves contacting the above residue with a mixture of esters solvent and hydrochloride. The ester is selected from the group consisting of ethyl acetate, isopropyl acetate and the like;

In sixth embodiment, the N-Boc Isavuconazonium chloride is purified by treatment with a solvent system comprising a mixture of methanol, water to obtain N-Boc Isavuconazonium chloride with a purity of at least 95%. The above process involves isolation by methods such as filtration, centrifugation.

In another embodiment conversion of N-Boc Isavuconazonium chloride to Isavuconazonium sulfate comprises: dissolving Isavuconazonium hydrochloride in methanol-water and treating with anion exchange resin to obtain Isavuconazonium sulfate.

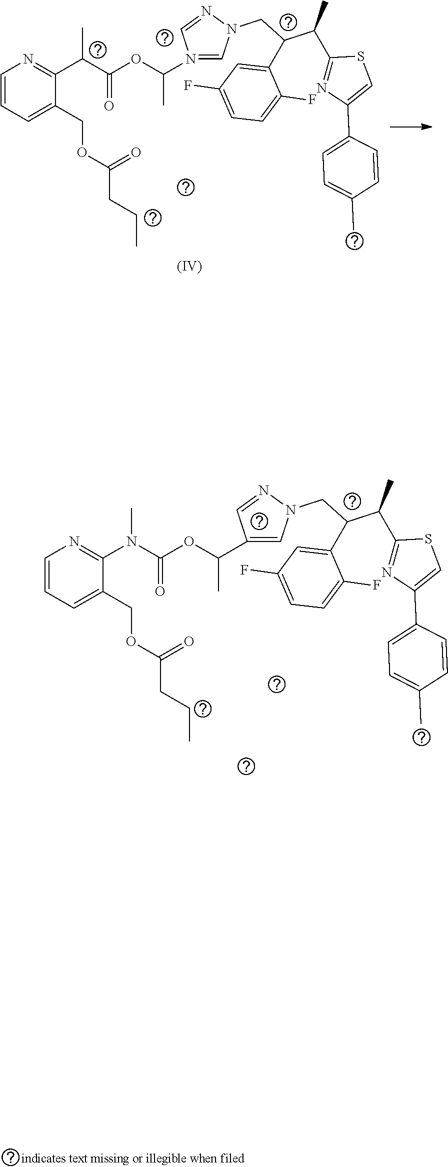

the amorphous Isavuconazonium sulfate obtained by the present invention has a chiral purity of at least 99%.

In another embodiment of the present invention provides a process for the preparation of Isavuconazole compound of formula KSM-I as illustrated in scheme 1.

In an illustrated embodiment, the Isavuconazole compound of formula KSM-I, which includes steps of a) reacting compound of formula 1 with compound formula 2 in presence of ethanol and base to obtain reaction mixture containing Isavuconazole.

Schematic representation of Scheme-2 is depicted below:

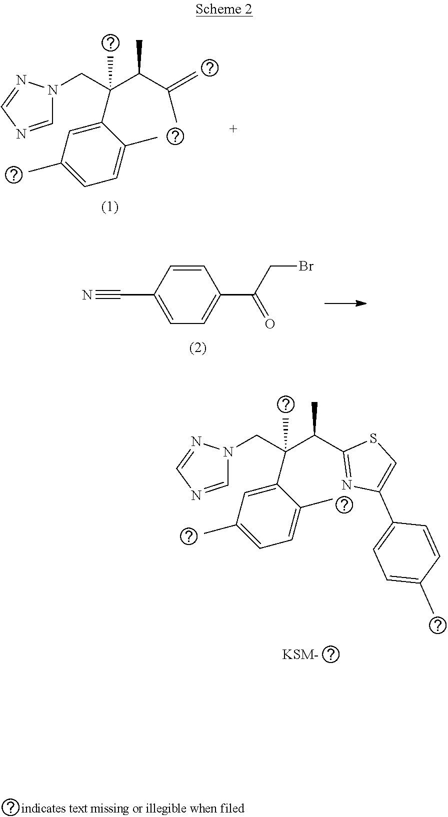

The reaction is carried at a temperature of about 20° C. to about reflux temperature of the solvent. Preferably the reaction is carried out at a temperature of about 45° C. to about 70° C. the reaction carried out in presence single solvent ethanol and base triethylamine.

The use of single organic solvent such as methanol in both reaction and workup results in a cost effective process.

In another embodiment, the reaction mixture containing Isavuconazole, the compound of formula KSM-I, is subjected to basification by a process comprising the step of adjusting the pH of the reaction mixture containing Isavuconazole to about 7 to about 8 at a temperature of about above 50° C.

In further embodiment, Isavuconazole from the reaction mixture is further isolated by a process comprising: i) extracting Isavuconazole from the reaction mixture by addition of ethanol to obtain a ethanolic solution comprising Isavuconazole; ii) heating the ethanolic solution comprising Isavuconazole; iii) adding water to the ethanolic solution of above step ii) to obtain an aqueous ethanolic solution comprising Isavuconazole; iv) cooling the above aqueous ethanolic solution comprising Isavuconazole; and v) isolating Isavuconazole.

In another embodiment of the present invention provides a process for the preparation of KSM-II as illustrated in Scheme 3.

### Schematic Representation of Scheme-3 is Depicted Below:

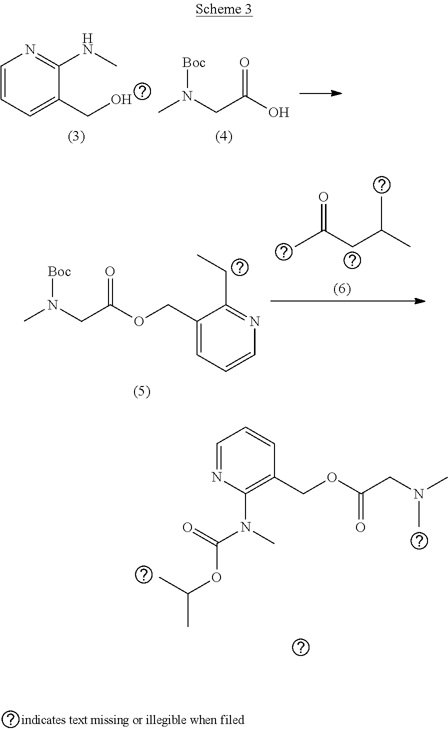 [0052] 1. In an illustrated embodiment, process for the preparation of KSM-II, which includes steps of reacting a compound of formula 3 with compound of formula 4 in presence of coupling reagent and solvent dichloromethane, base dimethyl amino pyridine at −5-10° C. to obtain compound of formula 5. In aspects, the suitable coupling reagent used includes but not limited to dicyclohexylcarbodiimide (DCC), 1,3-Diisopropylcarbodiimide (DIC) and 1-Ethyl-3-(3-dimethylaminopropyl)carbodiimide (EDCI).

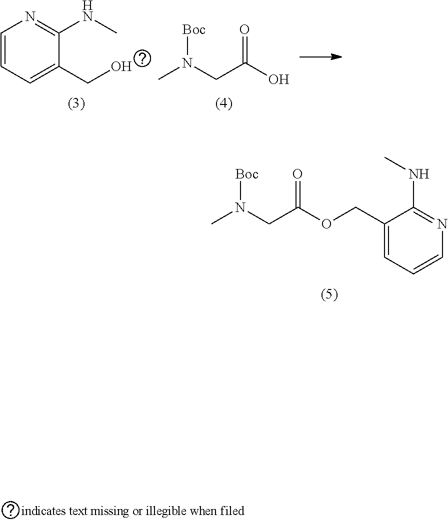

In further embodiment, the compound of formula 5 with protecting group 1-chloroethyl carbonochloridate (6) in presence of di isopropyl ethylamine dichloromethane at 20 to 40° C. to obtain compound formula KSM-II.

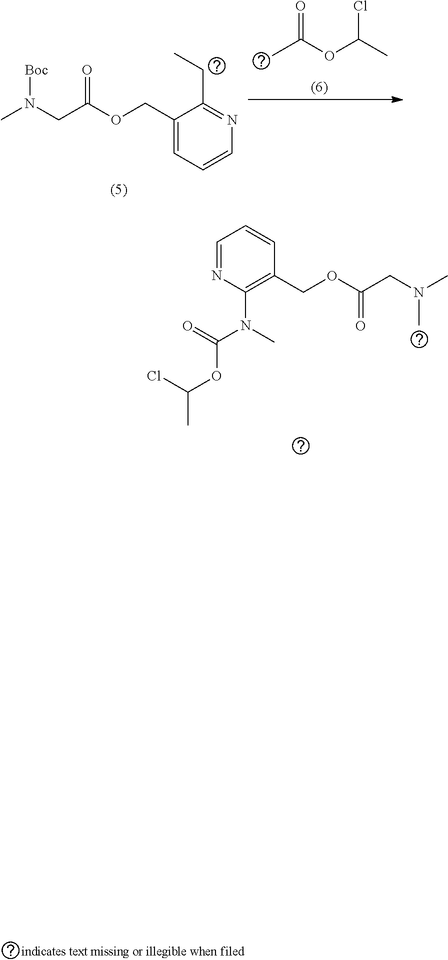

The present invention is further illustrated by the following example, which does not limit the scope of the invention. Certain modifications and equivalents will be apparent to those skilled in the art and are intended to be included within the scope of the present application.

### Examples

### Example 1: 4-{2-[(2R,3R)-3-(2,5-difluorophenyl)-3-hydroxy-4-(1H-1,2,4-triazoll-yl) butan-2-yl]-1,3-thiazol-4-yl} benzo nitrile (KSM-I)

To (2R,3R)-3-(2,5-difluorophenyl)-3-hydroxy-2-methyl-4-(1H-1,2,4-triazol-1-5yl) butane-thioamide (1) (100 g) in 500 mL ethanol was added 4-(bromoacetyl)benzo nitrile (2) (75 g). Reaction mass was heated to 55-65° C. After completion of reaction, pH of reaction mass adjusted to about 7.0 at 50-65° C. The layers were separated at a temperature of about 55° C. to about 60° C. The oily layer was dissolved in 400 mL ethanol and the obtained solution was heated to temperature of 10 about 55° C. to about 65° C. followed by addition of 1.5 volume of water. The reaction mass was cooled to temperature of about 20° C. to about 30° C. The solution was seeded with standard compound. The reaction mass was cooled to temperature of about 15° C. to about 20° C. and stirred for 8 hours. The solid was filtered and washed with a mixture of ethanol and water.

### Purity: 99%

### Example 2: 3-(tert-butoxycarbonylmethylamino) acetoxymethyl)-2-methylaminopyridine (5)

Dissolve 13.8 g (0.1 mol) 2-(N-methylamino)-3-hydroxymethylpyridine (3) and 23.5 g (0.125 mol) Boc-sarcosine (4) in 60 mL dichloromethane, slowly add 24 g at −7° C. (0.125 mol) EDCI and 3.66 g (0.03 mol) dimethylaminopyridine, after stirring for 2 h, the dichloromethane layer was washed with aqueous ammonium chloride solution, dried after separation, and the concentrated white solid was treated with DCM:PE=1:10 was recrystallized to obtain 28.5 g of a white solid.

### Yield: 92%

### Purity: 99.5%

### Example 3: Synthesis of [N-Methyl-N-3-((tert-butoxycarbonylmethylamino) acetoxymethyl) pyridin-2-yl] carbamic acid-1-chloroethyl ester (KSM-II)

Dissolve 30.9 g (0.1 mol) 3-(tert-butoxycarbonylmethylamino)acetoxymethyl)-2-methylamino pyridine (5) and 16.8 g (0.13 mol) diisopropylethylamine in 60 mL dichloromethane, 18.6 g (0.13 mol) chloroethyl chloroformate (6) was added dropwise at room temperature, the temperature was raised to 40° C., stirred for 2 h, cooled, washed with ammonium chloride aqueous solution, and the dichloromethane layer was separated, dried, and concentrated to obtain a yellow oil, dissolve it in 5 times the volume of ethyl acetate, add 1eq of ethyl acetate solution of hydrogen chloride dropwise at −5˜0° C., stir for 30 minutes, and filter to obtain 33.3 g of white solid.

### Yield: 80%.

### Purity: 87%.

### Example-4: Synthesis of 1-[[N-Methyl-N-3-[(t-butoxycarbonylmethylamino) acetoxymethyl] pyridin-2-yl] carbamoyloxy] ethyl-1-[(2R,3R)-2-(2,5-difluorophenyl)-2-hydroxy-3-[4-(4-cyanophenyl) thiazol-2-yl] butyl]-1H-[1,2,4]-triazo-4-ium iodide (Compound of formula III)

To a solution of [N-Methyl-N-2-(tert-butoxycarbonylisopropylamino) methyl) phenyl]carbamic acid 1-chloro-ethyl ester (KSM-II) (19.9 g. 48.0 mmol) in acetonitrile (100 ml) was added the azole compound (KSM-I) (15.0 g. 34.2 mmol) and sodium iodide (7.96 g. 48.0 mmol) at 70° C. The reaction mass was stirred for 6-8 hours at around 60-70° C. After completion of reaction, the solvent was removed and extracted with ethyl acetate. The organic phase was washed with water and brine. The solvent was removed in vacuo. The residue was purified by column chromatography (ethyl acetate to 10% methanol-dichloromethane) to afford 1-[[N-Methyl-N-3-[(t-butoxycarbonylmethylamino)acetoxymethyl]pyridin-2-yl] carba-moyloxy] ethyl-1-[(2R,3R)-2-(2,5-difluorophenyl)-2-hydroxy-3-[4-(4-cyanophenyl) thiazol-2-yl] butyl]-1H-[1,2,4]-triazo-4-ium iodide.

### Purity: 99%

### Example-5: Synthesis of 1-[[N-Methyl-N-3-[(methylamino)acetoxymethyl] pyridin-2-yl]carbamoyloxy] ethyl-1-[(2R,3R)-2-(2,5-difluorophenyl)-2-hydroxy-3-[4-(4-cyanophenyl) thiazol-2-yl] butyl]-1H-[1,2,4]-triazo-4-ium iodide hydrochloride (Compound of formula IV)

To a solution (100 ml) of 1-I[N-Methyl-N-2-(t-butoxycarbonylisopropylamino-methyl) phenyl] carbamoyloxylethyl-1-{(2R,3R)-2-(2,5-difluoropheny)-2-hydroxy-3-[4-(4-cyano-phenyl) thiazol-2-ylbutyl]-IH-|1,2,4) triazol-4-ium chloride (III) (14.8 g, 15.6 mmol) in ethyl acetate (150 ml) was added 4M HCl in ethyl acetate solution (60.0 ml) below 0-5° C. Gradually raised the temperature of reaction mass to room temperature. After stirring for 2 hours, the precipitate was filtered and washed with ethyl acetate. The precipitate was dried under vacuum to afford 1-I[N-methyl-N-2-(isopropylamino methy)phenyl] carbamoyloxylethyl-1-((28,3R)-2-(2,5-difluorophenyl)-2-hydroxy-3-[4(4-cyanophenyl)thiazol-2-yl] butyll-1H|1,2,4)triazol-4-ium chloride hydrochloride (13.70 g 18.0 mmol, quant) as off-white to yellow amorphous powder. Physical form: off-white to yellow amorphous powder.

### Purity: 99%

### Example 6: Process for the Preparation of Isavuconazonium Sulfate (V)

Dissolved 10.0 g 1-[[N-Methyl-N-3-[(methylamino)acetoxymethyl] pyridin-2-yl]carbamoyloxy] ethyl-l-[(2R,3R)-2-(2,5-difluorophenyl)-2-hydroxy-3-[4-(4-cyanophenyl) thiazol-2-yl] butyl]-1H-[1,2,4]-triazo-4-ium iodide hydrochloride (III) (10.0 g, 11.0 mmol) in Dichloromethane (50 ml) and demineralized water (25 mL). The solution was cooled to about 0 to 5° C. Slowly added 5% NaHCO₃ solution and adjusted pH between 7.0-7.7. Stirred the reaction mass for 45-60 minutes at 0-5° C. Separate the Dichloromethane layer and charged in another RB flask containing Deionized water (50 mL) and Diaion™ SA10A resin (50.0 g) was added to the cooled solution. The reaction mixture was stirred to about 60-80 minutes. The reaction was filtered and extracted. The aqueous layer was washed with 50 ml of Dichloromethane. The aqueous layer was lyophilized to obtain (8.0 g) white solid. Purity by HPLC: >99.0%, Isavuconazole impurity: ˜0.50%. Obtained product Isavuconazonium Sulfate is in Amorphous Form.

### Yield: 80%

### Purity: >99.0%

### Example 7: Process for the Preparation of Isavuconazonium Sulfate (V)

Dissolved 10.0 g 1-[[N-Methyl-N-3-[(methylamino)acetoxymethyl] pyridin-2-yl]carbamoyloxy] ethyl-1-[(2R,3R)-2-(2,5-difluorophenyl)-2-hydroxy-3-[4-(4-cyanophenyl) thiazol-2-yl] butyl]-1H-[1,2,4]-triazo-4-ium iodide hydrochloride (III) (10.0 g, 11.0 mmol) in Dichloromethane (50 ml) and demineralized water (25 mL). The solution was cooled to about 0 to 5° C. Slowly added 5% NaHCO₃ solution and adjusted pH between 7.0-7.7. Stirred the reaction mass for 45-60 minutes at 0-5° C. Separate the Dichloromethane layer and charged in another RB flask containing Deionized water (50 mL) and Diaion™ PA308 resin (50.0 g) was added to the cooled solution. The reaction mixture was stirred to about 60-80 minutes. The reaction was filtered and extracted. The aqueous layer was washed with 50 ml of Dichloromethane. The aqueous layer was lyophilized to obtain (8.0 g) white solid. Purity by HPLC: >99.0%, Isavuconazole impurity: ˜0.50%. Obtained product Isavuconazonium Sulfate is in Amorphous Form.

### Yield: 85%

### Purity: >99.0%

Without wishing to be bound to a theory, the process described in the present invention is believed to be an improved process for the preparation of Isavuconazonium Sulfate, which is commercially scalable, economical, stable and provides an improved yield along with high purity.

While the illustrative embodiments of the invention have been described with particularity, it will be understood that various other modifications will be apparent to and can be readily made by those skilled in the art without departing from the spirit and scope of the invention. Including all features which would be treated as equivalents thereof by those skilled in the art to which the invention pertains.

The present invention has the following advantages:
1. The prior art reported process require almost 15-16 hours, whereas the present invention process requires only 2-4 hours.
2. The anion exchange resins Diaion™ SA10A and Diaion™ PA308 used in present invention is cheaper than other available resins.
3. The resins used in the present invention can re-use.
4. Present invention getting good yields and purities of intermediate compounds than compared to yield and purities of past inventions.
5. Present invention getting good yield and purity.
6. Present invention is echo friendly, robust, safe and commercially feasible process.

We claim:

1. A process for the preparation of Isavuconazonium sulfate (V) having a purity of at least 99.5% by HPLC, which comprising:
a) reaction of (2R,3R)-3-(2,5-difluorophenyl)-3-hydroxy-2-methyl-4-(1H-1,2,4-triazol-1-5yl) butane-thioamide (1) with 4-(bromoacetyl)benzonitrile (2) in presence of suitable solvent and suitable base to obtain 4-{2-[(2R,3R)-3-(2,5-difluorophenyl)-3-hydroxy-4-(1H-1,2,4-triazoll-yl) butan-2-yl]-1,3-thiazol-4-yl} benzo nitrile (KSM-1); 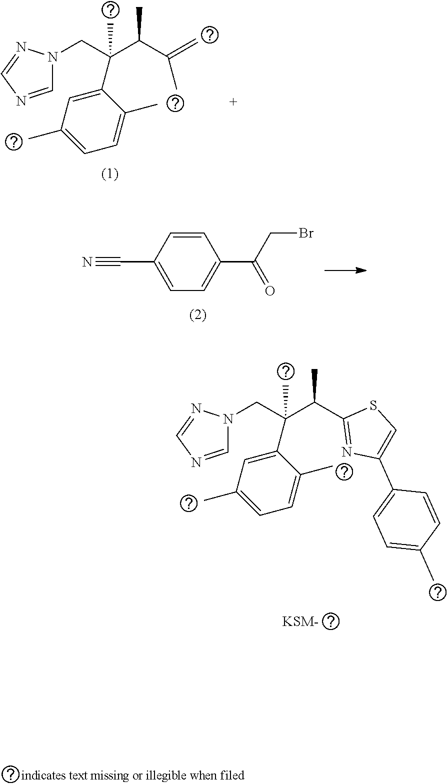
b) reaction of 2-(N-methylamino)-3-hydroxymethylpyridine (3) with Boc-sarcosine (4) in presence of suitable coupling reagent, suitable solvent and suitable base to obtain 3-(tert-butoxycarbonylmethylamino) acetoxymethyl)-2-methylaminopyridine (5); 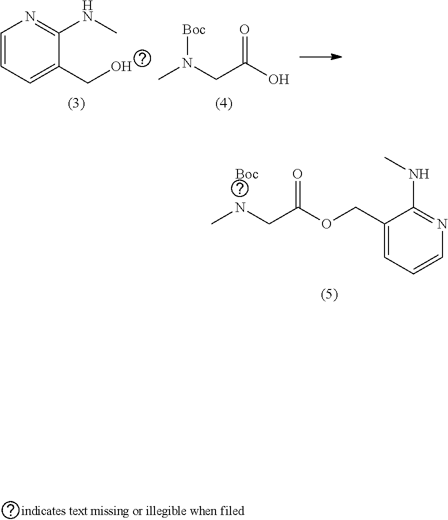
c) reaction of 3-(tert-butoxycarbonylmethylamino) acetoxymethyl)-2-methylaminopyridine (5) obtained in step b) with chloroethyl chloroformate (6) in presence of suitable solvent and suitable base to obtain [N-Methyl-N-3-((tert-butoxycarbonylmethylamino) acetoxymethyl) pyridin-2-yl] carbamic acid-1-chloroethyl ester (KSM-II); 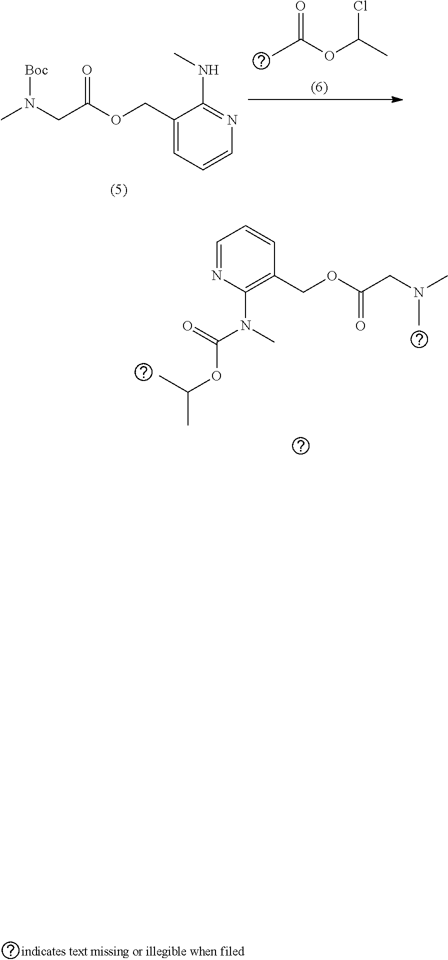
d) reaction of 4-{2-[(2R,3R)-3-(2,5-difluorophenyl)-3-hydroxy-4-(1H-1,2,4-triazoll-yl) butan-2-yl]-1,3-thiazol-4-yl} benzo nitrile (KSM-1) obtained in step a) with [N-Methyl-N-3-((tert-butoxycarbonylmethylamino) acetoxymethyl) pyridin-2-yl]carbamic acid-1-chloroethyl ester (KSM-II) obtained in step c) in presence of iodide salt, in suitable solvent to obtain 1-[[N-Methyl-N-3-[(t-butoxycarbonylmethylamino) acetoxymethyl] pyridin-2-yl] carbamoyloxy] ethyl-1-[(2R,3R)-2-(2,5-difluorophenyl)-2-hydroxy-3-[4-(4-cyanophenyl) thiazol-2-yl] butyl]-1H-[1,2,4]-triazo-4-ium iodide (III); 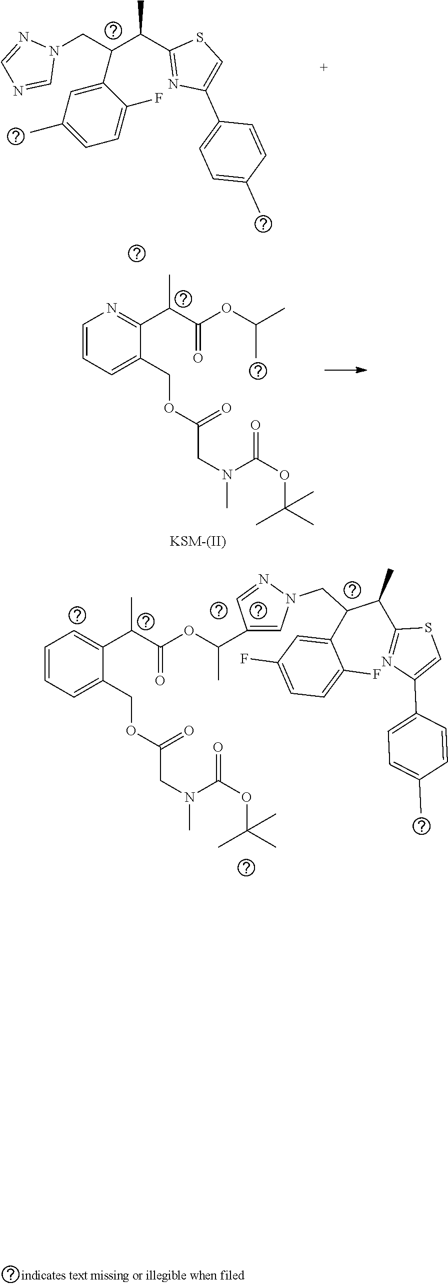
e) conversion of 1-[[N-Methyl-N-3-[(t-butoxycarbonylmethylamino) acetoxymethyl]pyridin-2-yl] carbamoyloxy] ethyl-1-[(2R,3R)-2-(2,5-difluorophenyl)-2-hydroxy-3-[4-(4-cyanophenyl) thiazol-2-yl] butyl]-1H-[1,2,4]-triazo-4-ium iodide (III) obtained in step d) into 1-I[N-Methyl-N-2-(t-butoxycarbonylisopropylamino-methyl) phenyl]carbamoyloxylethyl-1-{(2R,3R)-2-(2,5-difluoropheny)-2-hydroxy-3-14-(4-cyano-phenyl) thiazol-2-ylbutyl]-IH-|1,2,4) triazol-4-ium chloride (IV) in presence of ester solvent and hydrochloride; and 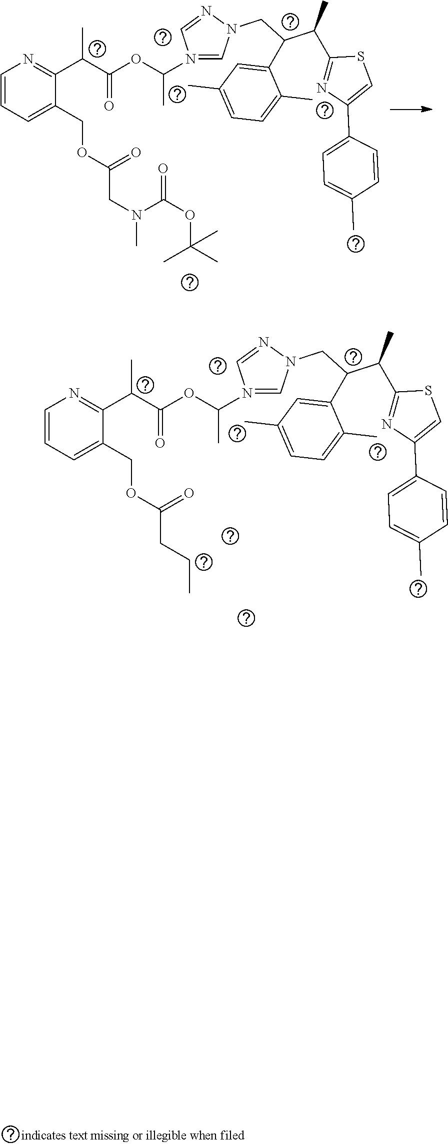
f) conversion of 1-I[N-Methyl-N-2-(t-butoxycarbonylisopropylamino-methyl) phenyl]carbamoyloxylethyl-1-{(2R,3R)-2-(2,5-difluoropheny)-2-hydroxy-3-14-(4-cyano-phenyl) thiazol-2-ylbutyl]-IH-|1,2,4) triazol-4-ium chloride (IV) obtained in step e) into Isavuconazonium sulfate (V) using anion exchange resin selected from Diaion™ SA10A or Diaion™ PA308 and solvent-water system.

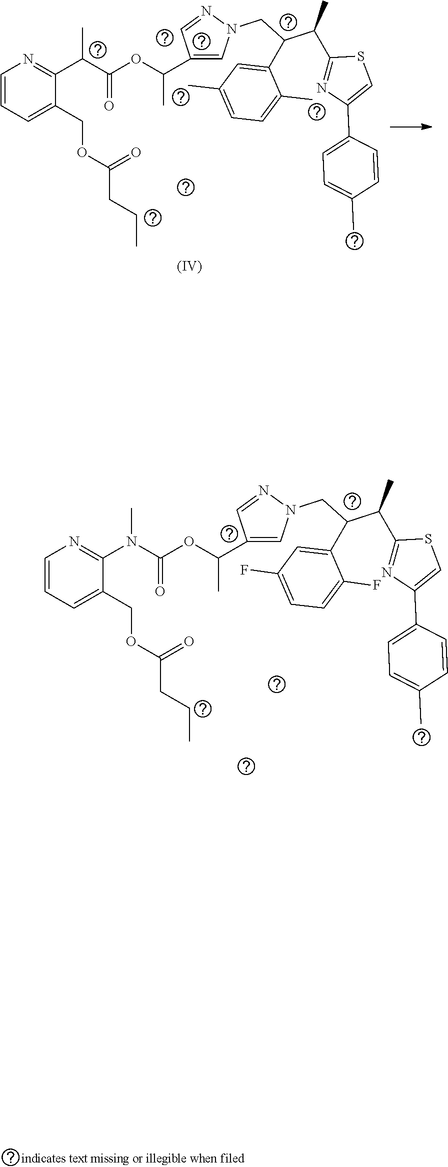

2. The process as claimed in claim 1, wherein the suitable solvent used in step a) to step f) is selected from water, methanol, ethanol, propanol, isopropanol, n-butanol, dichloromethane, dichloroethane, chloroform, carbon tetrachloride, dimethylacetamide (DMA), dimethylformamide (DMF), dimethylsulfoxide (DMSO), N-methylpyrrolidone (NMP), n-hexane, n-heptane, cyclohexane, toluene, pentane, cycloheptane, methyl cyclohexane and m-, o-, or p-xylene, acetonitrile and ethyl acetate or mixtures thereof.

3. The process as claimed in claim 1, wherein the suitable base used in step a) to step c) selected from the group consisting of organic base or inorganic base. The organic base is selected from Diisopropyl ethylamine (DIPEA), triethylamine, pyridine, 4-dimethylamino pyridine (DMAP) and the inorganic base is selected from the group comprising of sodium carbonate, potassium carbonate, lithium carbonate, calcium carbonate, cesium carbonate, magnesium carbonate, sodium hydroxide, potassium hydroxide, lithium hydroxide, calcium hydroxide, cesium hydroxide, magnesium hydroxide and the like, preferably triethyl amine, 4-dimethylamino pyridine (DMAP), sodium carbonate, potassium carbonate and sodium hydroxide.

4. The process as claimed in claim 1, wherein the suitable coupling reagent used in step b) is selected from the group consisting of dicyclohexylcarbodiimide (DCC), 1,3-Diisopropylcarbodiimide (DIC) and 1-Ethyl-3-(3-dimethylaminopropyl)carbodiimide (EDCI).

5. The process as claimed in claim 1, wherein the iodide salt used in step d) is selected from the group consisting of potassium iodate, potassium iodide, sodium iodate, and sodium iodide.
